(19) United States
(12) Patent Application Publication
(10) Pub. No.: US 2018/0066070 A1
(43) Pub. Date: Mar. 8, 2018

(54) ACETYLATED PCSK9

(71) Applicant: UNIVERSITAT ZURICH, Zurich (CH)

(72) Inventors: Melroy Xavier MIRANDA, Schlieren (CH); Thomas F. LUSCHER, Zurich (CH)

(73) Assignee: UNIVERSITAT ZURICH, Zurich (CH)

(21) Appl. No.: 15/551,601

(22) PCT Filed: Feb. 17, 2016
(86) PCT No.: PCT/EP2016/053399

§ 371 Date: Aug. 17, 2017

(30) Foreign Application Priority Data
Feb. 18, 2015 (EP) 15155667.7

(51) Int. Cl. C07K 16/44; A61K 39/395; C07K 16/40

(52) U.S. Cl. CPC C07K 16/44; A61K 2039/505; C07K 16/40; A61K 39/3955

(57) ABSTRACT

The invention relates to antibodies reactive to acetylated PCSK9, particularly to PCSK9 acetylated in position Lys421, Lys273, Lys69, Lys83, Lys243, Lys258, Lys494, and/or Lys506, and related reagents. The invention also relates to a method of reducing LDL-cholesterol level in a patient in need thereof, said method comprising administering to the subject an effective amount of an antibody binding to acetylated PCSK9. The invention also relates to a method of treating a cholesterol related disorder in a patient in need thereof, said method comprising administering to the subject an effective amount of an antibody binding to acetylated PCSK9. The invention also relates to a method of treating hypercholesterolemia in a patient in need thereof, said method comprising administering to the subject an effective amount of an antibody binding to acetylated PCSK9.

Proprotein convertase subtilisin-kexin type 9 (PCSK9) is a secreted protein that regulates serum LDL cholesterol by modulating hepatic LDL-receptor (LDLR) expression. Secreted PCSK9 binds to membrane bound-LDLR and LDL-complex and directs LDLR for lysosomal degradation rather than the recycling pathway. PCSK9 is initially synthesized as a 74 kDa proprotein, which undergoes autocatalytic cleavage of the 14 kDa N-terminal prodomain. The prodomain does not detach from the resulting 68 kDa mature form, but blocks the catalytic domain allowing PCSK9 to function as a chaperone. Inhibition of PCSK9 causes an increase in LDLR expression and thus reduces plasma LDL-cholesterol levels. Furthermore a reduced secretion of PCSK9 from the liver leads to reduced LDL-cholesterol in the plasma (Miranda et al., EurHeartJ, Mar. 6, 2014). Therefore altering the secretion of PCSK9 is a novel therapeutic target for the treatment of cholesterol related diseases and disorders. However, this therapeutic strategy has up to date not been available due to a lack of understanding of how PCSK9 secretion is regulated.

The problem underlying the present invention is to identify modulators of PCSK9 secretion, and agents interfering with the biological effects of PCSK9, for use in in vitro and in vivo therapeutic and diagnostic applications. This problem is solved by the subject matter of the independent claims.

### Terms and Definitions

Amino acid sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids.

In the context of the present specification, the term antibody is used in its meaning known in the art of cell biology and immunology. In particularly, the term refers to whole antibodies, any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxyterminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system.

In the context of the present specification, the term antigen binding portion or antigen binding fragment is used in its meaning known in the art of cell biology and immunology; it refers to one or more fragments of an intact antibody that retain the ability to specifically bind to a given antigen (e.g., acetylated PCSK9). Antigen binding functions of an antibody can be performed by fragments of an intact antibody. Examples of binding fragments encompassed within the term antigen binding portion or antigen binding fragment of an antibody include a Fab fragment, a monovalent fragment consisting of the V_{L}, V_{H}, C_{L} and C_{H} domains; a F(ab)₂ fragment, a bivalent fragment comprising two Fab fragments linked by a disulfide bridge at the hinge region; an Fd fragment consisting of the V_{H} and C_{H} domains; an Fv fragment consisting of the V_{L} and V_{H} domains of a single arm of an antibody; a single domain antibody (dAb) fragment, which consists of a V_{H} domain or a V_{L} domain; and an isolated complementarity determining region (CDR).

In the context of the present specification, the term chimeric antibody is used in its meaning known in the art of cell biology and immunology; it refers to an antibody molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, cytokine, toxin, hormone, growth factor, drug, etc. For example, an antibody can be modified by replacing its constant region with a cytokine. Due to the replacement with a cytokine, the chimeric antibody can retain its specificity in recognizing the antigen while having also the function, or part thereof, of the original cytokine molecule.

In the context of the present specification, the term hybridoma is used in its meaning known in the art of cell biology and biochemistry; it refers to a hybrid cell created by fusion of a specific antibody-producing B-cell with a myeloma (B-cell cancer) cell. Hybridoma cells can be grown in tissue culture and produce antibodies of a single specificity (monoclonal antibodies).

In the context of the present specification, the term single-chain variable fragment (scFv) is used in its meaning known in the art of cell biology and biochemistry; it refers to a fusion protein of the variable regions of the heavy (V_{H}) and light chains (V_{L}) of immunoglobulins, connected with a short linker peptide of ten to about 25 amino acids. The scFv retains the specificity of the original immunoglobulin, despite removal of the constant regions and the introduction of the linker.

In the context of the present specification, the term fragment antigen-binding (Fab) is used in its meaning known in the art of cell biology and immunology; it refers to a region on an antibody that binds to antigens. It is composed of one constant and one variable domain of each of the heavy (V_{H}) and light chains (V_{L}) of immunoglobulins. These domains shape the antigen-binding site at the amino terminal end of the monomer.

In the context of the present specification, the term humanized antibodies is used in its meaning known in the art of cell biology and biochemistry; it refers to antibodies originally produced by immune cells of a non-human species, whose protein sequences have been modified to increase their similarity to antibody variants produced naturally in humans.

In the context of the present specification, the term dissociation constant (K_{D}) is used in its meaning known in the art of chemistry and physics; it refers to an equilibrium constant that measures the propensity of a larger object to dissociate reversibly into smaller components, as when a complex falls apart into its component molecules. K_{D} is expressed in molar units [M] and corresponds to the concentration of [Ab] at which the binding sites of [Ag] are half occupied. In other words the concentration of unbound [Ab] equals the concentration of the [AbAg] complex. The dissociation constant can be calculated according to the following formula:

$${K}_{D} = \frac{[ Ab ] * [ Ag ]}{[ AbAg ]}$$

[Ab]: concentration of antibody; [Ag]: concentration of antigen; [AbAg]: concentration of antibody-antigen complex

In the context of the present specification, the terms off-rate (K_{off}; [1/sec]) and on-rate (Kₒₙ; [1/sec*M]) are used in their meaning known in the art of chemistry and physics; they refer to a rate constant that measures the dissociation (K_{off}) or association (Kₒₙ) of an antibody with its target antigen. K_{off} and Kₒₙ can be experimentally determined using methods well established in the art. A method for determining the K_{off} and Kₒₙ of an antibody employs surface plasmon resonance. This is the principle behind biosensor systems such as the Biacore® or the ProteOn® system. They can also be used to determine the dissociation constant K_{D} by using the following formula:

$${K}_{D} = \frac{{K}_{off}}{{K}_{on}}$$

In the context of the present specification the term virus like particle (VLP) refers to a structure resembling a virus particle, but which has been demonstrated to be non-pathogenic. In general, virus-like particles lack at least part of the viral genome. A virus-like particle may contain nucleic acid distinct from their genome. A typical embodiment of a virus-like particle in is a viral capsid such as the viral capsid of the corresponding virus, bacteriophage, or RNA-phage.

In the context of the present specification the term immunogen refers to an antigen that is capable of inducing a humoral and/or cell-mediated immune response.

In the context of the present specification the term immunogenic carrier includes those materials which have the property of independently eliciting an immunogenic response in a host animal and which can be covalently coupled to a peptide, polypeptide or protein either directly via formation of peptide or ester bonds between free carboxyl, amino or hydroxyl groups in the peptide, polypeptide or protein and corresponding groups on the immunogenic carrier material, or alternatively by bonding through a conventional bifunctional linking group, or as a fusion protein.

In the context of the present specification the term proprotein convertase subtilisin kexin type 9 (PCSK9), refers to any native PCSK9 from any vertebrate source, including mammals such as primates (e.g. humans) and rodents (e.g., mice and rats), unless otherwise indicated. The term encompasses full-length unprocessed PCSK9 as well as any form of modified, particularly acetylated, PCSK9 that results from processing in the cell or any fragment thereof. The term also encompasses naturally occurring variants of PCSK9, e.g., splice variants or allelic variants.

In the context of the present specification the term Sirtuin (silent mating type information regulation 2 homolog) 1 is equivalently used to the term SIRT1. Sirtuin 1 is a highly conserved protein deacetylase that requires NAD (nicotinamide adenine dinucleotide) as a co-substrate. The deacetylation of acetyl-lysines by Sirtuin 1 is coupled with NAD hydrolysis, producing nicotinamide and an acetyl-ADP ribose compound. Sirtuin 1 also exhibits NAD-dependent histone deacetylase activity.

In the context of the present specification the term pCAF refers to the p300/CBP-associated factor. pCAF has acetyltransferase and E3 ubiquitin ligase domains as well as a bromodomain for interaction with other proteins.

In the context of the present specification the terms acetylation and deacetylation are used in their meaning known in the art of biochemistry and cell biology; it refers to a modification of proteins, where acetyl groups are covalently attached to or removed from lysine residues within the protein (see scheme 1). This modification is known to affect the properties and functions of the proteins. Acetylation of proteins is catalyzed by acetyltransferases, whereas deacetylation is catalyzed by deacetylases. One example of an acetyltransferase is p300/CBP-associated factor (pCAF) and one example of a deacetylase is Sirtuin 1 (SIRT1).

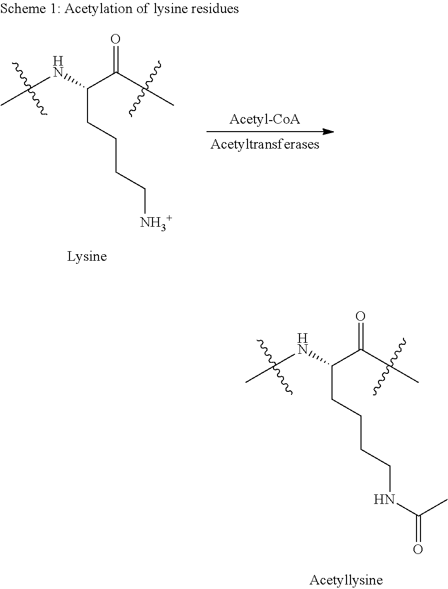 [SMILES: *N[C@@H](CCCC[NH3+])C(*)=O ; *N[C@@H](CCCCNC(C)=O)C(*)=O]

In the context of the present specification the term high-density lipoprotein (HDL) relates to a class of plasma lipoproteins with a high proportion of protein, including apolipoproteins A, C, D and E. HDL incorporates and transports cholesterol, whether free or esterified, in the plasma as an HDL-cholesterol complex. The term HDL may be used in a context-dependent manner to designate cholesterol bound to HDL particles.

In the context of the present specification the term low-density lipoprotein (LDL) relates to a class of plasma lipoproteins with a high proportion of lipid, including cholesterol, cholesterol esters and triglycerides. It includes primarily apolipoprotein B-100 and apolipoprotein E. LDL incorporates and transports cholesterol in the plasma. The term LDL may be used in a context-dependent manner to designate cholesterol bound to LDL particles.

In the context of the present specification the term cardiovascular disease (CVD) has its general meaning in the art and is used to classify conditions that affect the heart, heart valves, blood, and vasculature of the body. Cardiovascular diseases include endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm. Such diseases frequently involve atherosclerosis.

### SUMMARY OF THE INVENTION

According to a first aspect of the invention a ligand that binds to acetylated PCSK9, but not to non-acetylated PCSK9 is provided.

According to a second aspect of the invention, a ligand, particularly an isolated antibody, that binds to acetylated PCSK9 is provided. Binding of the ligand to acetylated PCSK9 is characterized by a first dissociation constant K_{D}1 whereas binding of the ligand to non-acetylated PCSK9 is characterized by a second dissociation constant K_{D}2. The relation of K_{D}1 and K_{D}2 is K_{D}1*100<K_{D}2.

In certain embodiments the relation of K_{D}1 and K_{D}2 is: K_{D}1*10<K_{D}2, K_{D}1*20<K_{D}2, K_{D}1*50<K_{D}2, K_{D}1*1000<K_{D}2 or K_{D}1*10000<K_{D}2.

In certain embodiments the first dissociation constant is: K_{D}1<10 nmol/l, K_{D}1<1 nmol/l or K_{D}1<0.1 nmol/l.

Sites of Acetylation on PCSK9

In certain embodiments of the first and second aspect of the invention the ligand, particularly the antibody, binds to an epitope on acetylated PCSK9 comprising an acetylated lysine residue Lys421.

In certain embodiments, the ligand, particularly the antibody, binds to PCSK9 acetylated in position Lys421, but not to PCSK9 that is not acetylated in position Lys421, particularly wherein the binding of the ligand or antibody to PCSK9 acetylated in Lys421 is characterized by a first dissociation constant K_{D}1 whereas binding of the ligand or antibody to non-acetylated PCSK9 in Lys421 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys273.

In certain embodiments, the ligand, particularly the antibody, binds to PCSK9 acetylated in position Lys273, but not to PCSK9 that is not acetylated in position Lys273, particularly wherein the binding of the ligand or antibody to PCSK9 acetylated in Lys273 is characterized by a first dissociation constant K_{D}1 whereas binding of the ligand or antibody to non-acetylated PCSK9 in Lys273 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys69.

In certain embodiments, the ligand, particularly the antibody, binds to PCSK9 acetylated in position Lys69, but not to PCSK9 that is not acetylated in position Lys69, particularly wherein the binding of the ligand or antibody to PCSK9 acetylated in Lys69 is characterized by a first dissociation constant K_{D}1 whereas binding of the ligand or antibody to non-acetylated PCSK9 in Lys69 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys83.

In certain embodiments, the ligand, particularly the antibody, binds to PCSK9 acetylated in position Lys83, but not to PCSK9 that is not acetylated in position Lys83, particularly wherein the binding of the ligand or antibody to PCSK9 acetylated in Lys83 is characterized by a first dissociation constant K_{D}1 whereas binding of the ligand or antibody to non-acetylated PCSK9 in Lys83 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys243.

In certain embodiments, the ligand, particularly the antibody, binds to PCSK9 acetylated in position Lys243, but not to PCSK9 that is not acetylated in position Lys243, particularly wherein the binding of the ligand or antibody to PCSK9 acetylated in Lys243 is characterized by a first dissociation constant K_{D}1 whereas binding of the ligand or antibody to non-acetylated PCSK9 in Lys243 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys258.

In certain embodiments, the ligand, particularly the antibody, binds to PCSK9 acetylated in position Lys258, but not to PCSK9 that is not acetylated in position Lys258, particularly wherein the binding of the ligand or antibody to PCSK9 acetylated in Lys258 is characterized by a first dissociation constant K_{D}1 whereas binding of the ligand or antibody to non-acetylated PCSK9 in Lys258 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys494.

In certain embodiments, the ligand, particularly the antibody, binds to PCSK9 acetylated in position Lys494, but not to PCSK9 that is not acetylated in position Lys494, particularly wherein the binding of the ligand or antibody to PCSK9 acetylated in Lys494 is characterized by a first dissociation constant K_{D}1 whereas binding of the ligand or antibody to non-acetylated PCSK9 in Lys494 is characterized by a second dissociation constant K_{D}2, and the relation of K_{D}1 and K_{D}2 is K_{D}1*100<K_{D}2.

In certain embodiments of the first and second aspect of the invention the ligand, particularly the antibody, binds to an epitope on acetylated PCSK9 comprising an acetylated lysine residue Lys506.

In certain embodiments, the ligand, particularly the antibody, binds to PCSK9 acetylated in position Lys506, but not to PCSK9 that is not acetylated in position Lys506, particularly wherein the binding of the ligand or antibody to PCSK9 acetylated in Lys506 is characterized by a first dissociation constant K_{D}1 whereas binding of the ligand or antibody to non-acetylated PCSK9 in Lys506 is characterized by a second dissociation constant K_{D}2, and the relation of K_{D}1 and K_{D}2 is K_{D}1*100<K_{D}2.

Antibody Based Scaffolds Targeting Acetylated PCSK9

In certain embodiments, said ligand that binds to acetylated PCSK9, but not to non-acetylated PCSK9 is an isolated antibody, or antigen binding fragment thereof.

In certain embodiments, said ligand that binds to acetylated PCSK9, but not to non-acetylated PCSK9 is a human antibody.

In certain embodiments, said ligand that binds to acetylated PCSK9, but not to non-acetylated PCSK9 is a humanized antibody.

In certain embodiments, said ligand that binds to acetylated PCSK9, but not to non-acetylated PCSK9 is a chimeric antibody.

In certain embodiments of the first and second aspect of the invention the antibody is a mouse, rabbit or goat antibody.

Non-Antibody Scaffolds Targeting Acetylated PCSK9

Alternative target binding ligand proteins have been proposed recently, which are more diverse in molecular structure than human immunoglobulin-derived antibody fragments and antibody-derived constructs and formats, and thus allow additional molecular formats by creating heterodimeric and multimeric assemblies, leading to new biological functions. A number of such target binding ligand proteins have been described (reviewed in (Binz et al., Nat. Biotech 2005, Vol 23:1257-1268)). Non-limiting examples of such target binding ligand proteins are camelid antibodies, protein scaffolds derived from protein A domains (termed “Affibodies”, Affibody AB), tendamistat (an alpha-amylase inhibitor, a 74 amino acid beta-sheet protein from Streptomyces tendae), fibronectin, lipocalin (“Anticalins”, Pieris), T-cell receptors, ankyrins (designed ankyrin repeat proteins termed “DARPins”, Univ. Zurich and Molecular Partners; see US20120142611 (A1)), A-domains of several receptors (“Avimers”, Avidia) and PDZ domains, fibronectin domains (FN3) (“Adnectins”, Adnexus), consensus fibronectin domains (“Centyrins”, Centyrex/Johnson&Johnson) and Ubiquitin (“Affilins”, SCIL Proteins) and knottins (Moore and Cochrane, Methods in Enzymology 503 (2012), 223-251 and references cited therein). All the above mentioned target binding ligand proteins are contemplated as embodiments of the ligand according to the invention that binds to acetylated PCSK9, but not to non-acetylated PCSK9.

From these target binding ligand proteins, multimeric and multispecific assemblies can be constructed (Caravella and Lugovskoy, Current Opinions in Chemical Biology 2010, 14:520-528; Vanlandschoot et al. Antiviral Research 2011 92:389-407; Lofblom et al. 2011 Current Opinion in Biotechnology 2011 22:843-848, Boersma et al. 2011 Curr. Opin. Biotechnol. 22:849-857). It is also possible to fuse these and other peptidic domains to antibodies to create so-called Zybodies (Zyngenia Inc., Gaithersburg, Md.).

All of these scaffolds, with different inherent properties, have in common that they can be directed to bind specific epitopes, by using selection technologies well known to practitioners in the field (Binz et al. ibid.).

Immobilized acetylated PCSK9 can serve as a target for diverse protein libraries in either phage display or ribosome display format. A large variety of different antibody libraries has been published (Mondon P. et al., Human antibody libraries: a race to engineer and explore a larger diversity. Frontiers in Bioscience. 13:1117-1129, 2008.) and the technology of selecting binding antibodies is well known to the practitioners of the field. Phage display is a suitable format for antibody fragments (Fab fragments, scFv fragments or single domain antibodies s) (Hoogenboom H R. Nature Biotechnology. 23(9):1105-1116, 2005 September) and any other scaffold that contain disulfide bonds, but it can also be used for scaffolds not containing disulfide bonds (e.g., Steiner et al. (2008) J. Mol. Biol. 382, 1211-1227; Rentero et al. Chimia. 65(11):843-5, 2011., Skerra A. Current Opinion in Biotechnology. 18(4):295-304, 2007 August). Similarly, ribosome display can be used for antibody fragments (Hanes et al. (2000), Picomolar affinity antibodies from a fully synthetic naive library selected and evolved by ribosome display. Nat. Biotechnol. 18, 1287-1292) and for other scaffolds (Zahnd et al. (2007). Ribosome display: selecting and evolving proteins in vitro that specifically bind to a target. Nat. Methods 4, 269-279; Zahnd et al. (2007) J. Mol. Biol. 369, 1015-28.). A third powerful technology is yeast display (Pepper et al., Combinatorial Chemistry & High Throughput Screening. 11(2):127-134, 2008 Feb.). In this case a library of the binding protein of interest is displayed on the surface of yeast, and the respective acetylated epitope of PCSK9 is either directly labeled with a fluorescent dye or its his tag is detected with an anti-histag antibody, which is in turn detected with a secondary antibody. Such methods are well known to the practitioners in the field (Boder et al., Yeast surface display for directed evolution of protein expression, affinity, and stability, Methods in Enzymology. 328:430-44, 2000.).

In certain embodiments, the ligand according to the first or second aspect of the invention, which binds to acetylated PCSK9, but not to non-acetylated PCSK9, is a designed ankyrin repeat protein (DARPin).

In certain aspects of the invention, any of the above mentioned ligands or antibodies are provided for use in a method for lowering cholesterol in a patient, or for prevention or treatment of cholesterol-related diseases exemplified by, but not limited to, atherosclerosis, endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm.

According to a third aspect of the invention a pharmaceutical composition is provided comprising the anti-acetylated PCSK9 ligand, particularly the antibody, according to the first or second aspect of the invention and a pharmaceutically acceptable carrier, particularly for use in a method for lowering cholesterol in a patient, or for prevention or treatment of cholesterol-related diseases exemplified by, but not limited to, atherosclerosis, endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm.

According to a fourth aspect of the invention a method of reducing LDL-cholesterol level in a subject is provided. The method comprises administering to the subject an effective amount of the anti-accetylated PCSK9 ligand, particularly the antibody, according to the first or second aspect of the invention.

According to a fifth aspect of the invention a method of treating cholesterol related disorder in a subject is provided. The method comprises administering to the subject an effective amount of the anti-acetylated PCSK9 ligand, particularly the antibody, according to the first or second aspect of the invention.

According to a sixth aspect of the invention a method of treating hypercholesterolemia in a subject is provided. The method comprises administering to the subject an effective amount of the anti-acetylated PCSK9 ligand, particularly the antibody, according to the first or second aspect of the invention.

Without wishing to be bound by theory, it is assumed that deacetylation of PCSK9 prevents its secretion from the liver and therefore only acetylated PCSK9 is secreted. This means that an antibody targeting acetyl-PCSK9 can have therapeutic value by binding a target outside the liver. Also quantification of acetyl-PCSK9 can be diagnostic.

According to a seventh aspect of the invention a method of treating a cholesterol related disorder in a subject is provided. The method comprises administering to the subject an effective amount of a substance that reduces PCSK9 acetylation levels in the liver.

Yet another aspect of the invention provides a nucleic acid molecule encoding the ligand or antibody according to the first and second aspect of the invention.

Another aspect of the invention relates to an expression cassette comprising the nucleic acid molecule of the invention under control of a promoter sequence operable in a mammalian cell.

Yet another aspect of the invention relates to a transgenic cell comprising an expression cassette according to the ninth aspect of the invention.

Another aspect of the invention relates to a cell able to produce a ligand, particularly an antibody, or antigen binding fragment thereof, according to the first and second aspect of the invention.

Another aspect of the invention relates to an isolated acetylated PCSK9 molecule. In certain embodiments the position of acetylation of the PCSK9 molecule is Lys421. In certain embodiments the position of acetylation of the PCSK9 molecule is Lys4273. In certain embodiments the position of acetylation of the PCSK9 molecule is Lys69. In certain embodiments the position of acetylation of the PCSK9 molecule is Lys83. In certain embodiments the position of acetylation of the PCSK9 molecule is Lys243. In certain embodiments the position of acetylation of the PCSK9 molecule is Lys258. In certain embodiments the position of acetylation of the PCSK9 molecule is Lys494. In certain embodiments the position of acetylation of the PCSK9 molecule is Lys506.

Another aspect of the invention relates to an immunogen comprising an acetylated-PCSK9 peptide linked to an immunogenic carrier. In certain embodiments the immunogenic carrier is selected from Diphtheria toxiod, CRM197 or a VLP selected from HBcAg, HBsAg, Qbeta, PP7, PPV or Norwalk Virus VLP.

Another aspect of the invention relates to a method of predicting response to treatment with a cholesterol lowering drug is provided. The level of acetylated-PCSK9 is measured in a sample obtained from a patient prior to the patient taking a cholesterol lowering drug or after a sufficient washout period has elapsed for the patient. If the measured acetylated-PCSK9 levels fall within a predetermined target range, this is indicative that the patient is likely to respond favourably to treatment with a cholesterol lowering drug. Whereas a measured acetylated-PCSK9 level above the predetermined range is indicative that the patient is likely to have an attenuated response to treatment with a cholesterol lowering drug.

In certain embodiments the cholesterol lowering drug is selected from the group of statins, or the group of PCSK9 inhibitors.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows PCSK9 acetylation in plasma samples. After immunoprecipitation of plasma samples with an anti-K421ac-PCSK9 antibody, aliquots of immunoprecipitated protein (25 μg/lane) were subjected to SDS-PAGE and blots analysed for acetyl-lysine and PCSK9 with an anti-acetyl lysine antibody (upper panel) or an anti-PCSK9 antibody (lower panel).

FIG. 2 shows that PCSK9 is deacetylated by SIRT1 in vitro. Recombinant PCSK9 was incubated with SIRT1 and with or without NAD+ for 1 hour. Aliquots were subjected to SDS-PAGE and blots were analysed for acetyl-lysine and PCSK9.

FIG. 3 shows that SIRT1 is bound to PCSK9 in HepG2 cells. PCSK9 was immunoprecipitated from HepG2 cells overexpressing pCAF and SIRT1 using pCMV-pCAF and pCMV-SIRT1 overexpression vectors, and from cells treated with vehicle (DMSO) and SIRT1 activator SRT1720. Aliquots of immunoprecipitated protein were subjected to SDS-PAGE and blots analysed for SIRT1, acetyl-lysine and PCSK9.

FIG. 4 shows that SIRT1 deacetylates fluorogenic PCSK9 peptide: Bar graph representing increase in fluorescence caused by deacetylation of fluorogenic PCSK9 peptide on increasing concentrations of SIRT1 activator SRT1720 compared to negative and positive controls.

FIG. 5 shows results of the mapping of PCSK9 lysine acetylation by tandem mass spectrometry. PCSK9 were immunoprecipitated from HepG2 cells and incubated with recombinant SIRT1 with and without NAD+. The protein from the Colloidal Coomassie stained band was digested with trypsin and the resulting peptides were analyzed by LC-MS/MS and MALDI MS/MS. MS/MS data were analyzed using the sequences of PCSK9 with the Mascot algorithm, allowing the detection of acetylated lysine residues (red). (A) MS/MS spectrum of all peptides recovered in the LC-MS/MS experiment for PCSK9, highlighted in blue (B-E) Identification of acetylation at K421, K273 K243 & K506 in PCSK9 by MALDI. The predicted fragment ions for the acetylated peptides are shown in Example 3.

FIG. 6 shows a Coomasie stain of acetylated and deacetylated PCSK9. PCSK9 immunoprecipitated from HepG2 cells were treated with recombinant SIRT1 and NAD+ or recombinant pCAF with acetyl-CoA and were separated by SDS-PAGE, stained with Coomassie blue and cut into small slices. Each gel slice was subjected to densitometry and MS analysis.test gel (Bis-Tris gradient gel 4-12%) for mass spectrometry analysis as explained in example 2.

FIG. 7 shows that acetyl-PCSK9 is more active in RAW 264.7 macrophages. Recombinant PCSK9 pretreated with SIRT1 and NAD+ or pCAF and acetyl-CoA were added on RAW 264.7 macrophages for 15 mins and lysed, separated by SDS-PAGE and blotted for LDLR, PCSK9 and b-actin. Representative bar graphs of LDLR, PCSK9 normalized to b-actin and PCSK9 expression normalized to LDLR expression.

FIG. 8 shows that acetylation of PCSK9 at Lys421 is required for secretion and activity. (A) HepG2 cells were transfected with empty vector, wild-type PCSK9 and deacetylation mimetic K421 R-PCSK9. Cells were lysed after 24 hrs, separated by SDS-PAGE and blotted for LDLR, PCSK9 and b-actin. (B) C57B16 mice were treated with acetyl-PCSK9 antibody targeting Lys421 and after a duration of 5 days liver lysates were denatured, separated by SDS-PAGE and blotted for LDLR, PCSK9 and b-actin.

FIG. 9 shows that inhibition of acetylated PCSK9 provides differential expression of LDLR and PCSK9 activity. C57BI6 mice were treated with acetyl-PCSK9 antibody targeting Lys421, Lys69, Lys83, Lys243, Lys258 or Lys494 and after a duration of 5 days liver lysates were denatured, separated by SDS-PAGE and blotted for LDL, PCSK9 and b-actin. Representative bar graph showing relative LDLR expression, normalized to b-actin.

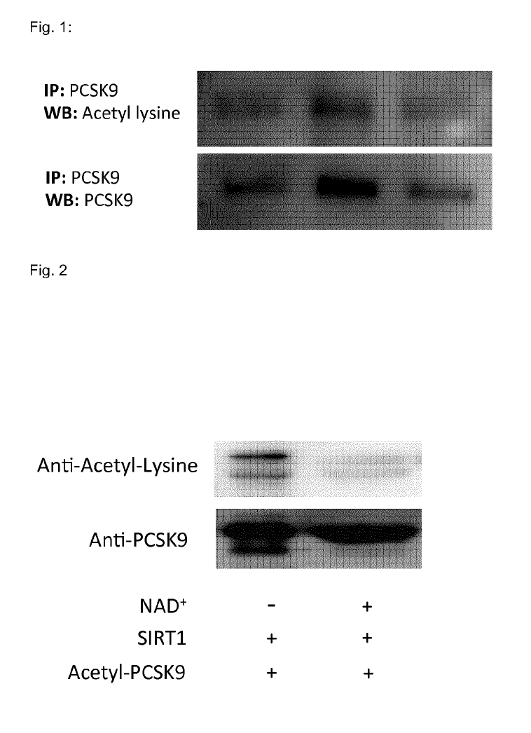

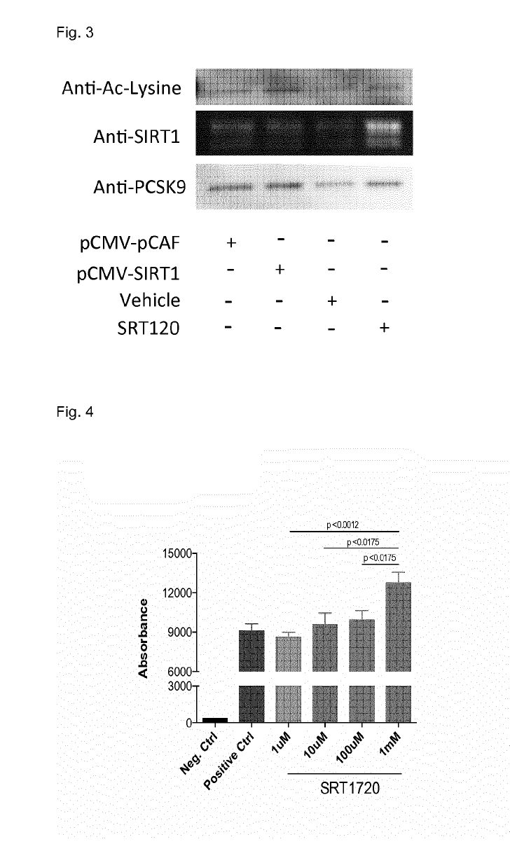

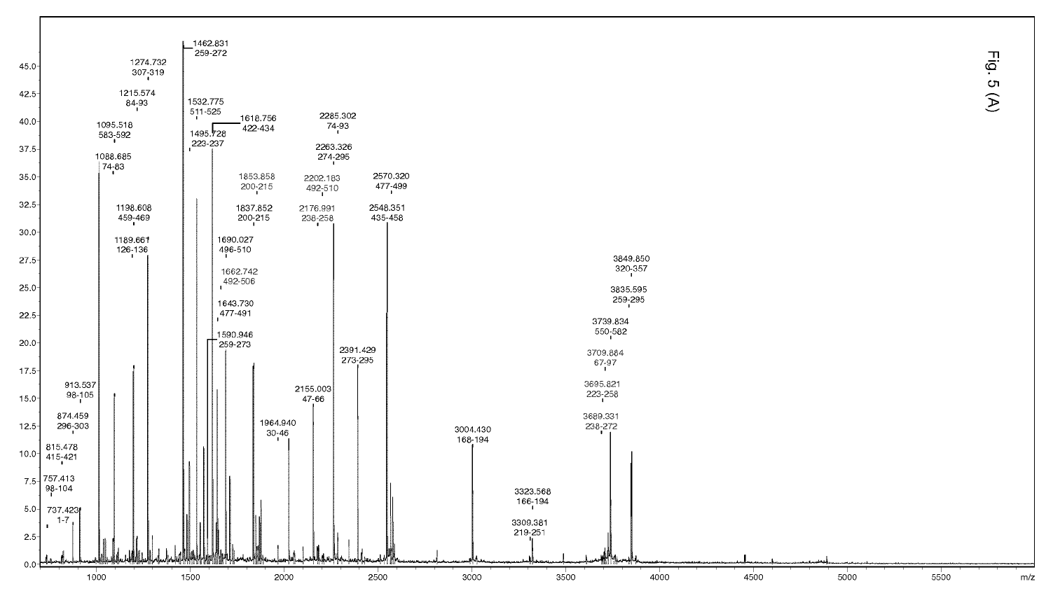

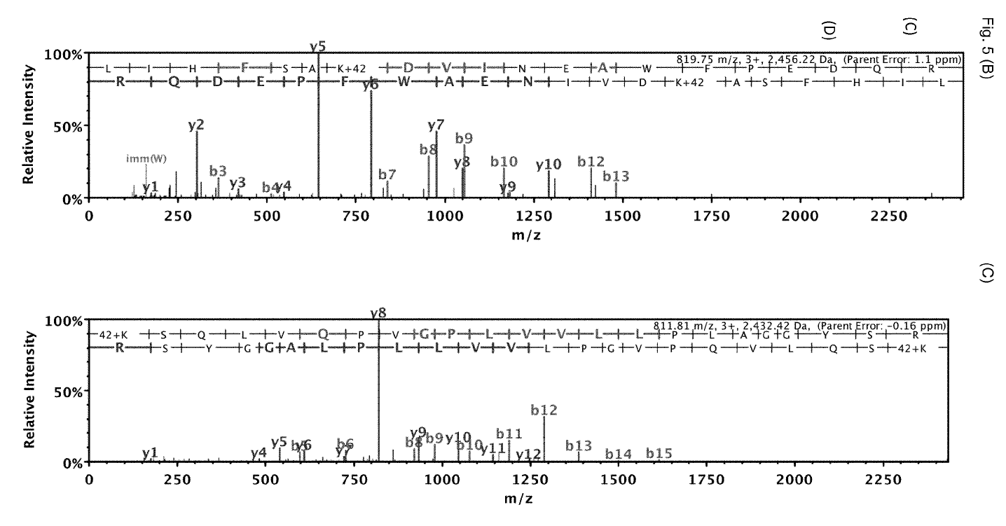

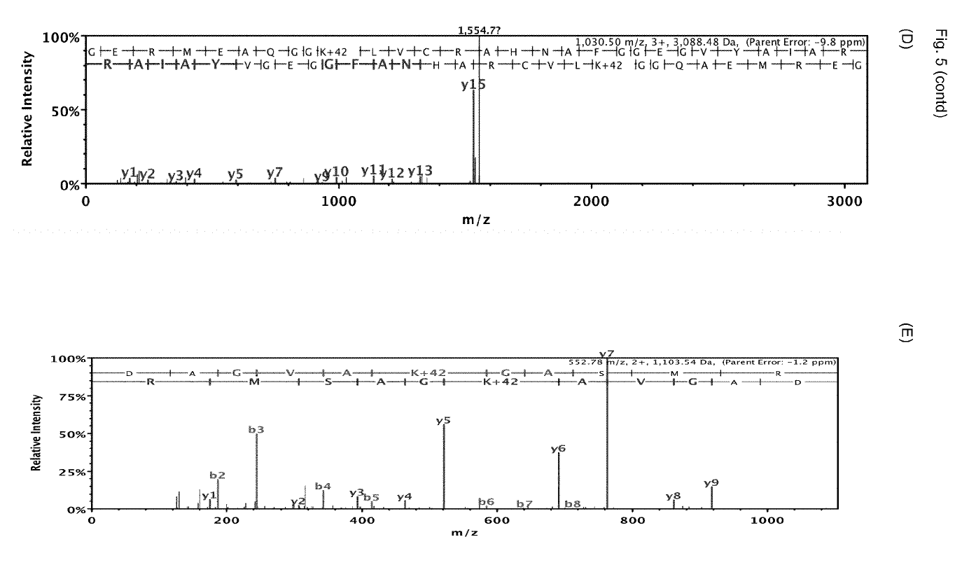

### EXAMPLES

It is known in the art that SIRT1 activation by SRT3025 in ApoE^{-/-} mice protects against atherosclerosis by reducing LDL-cholesterol through a reduction in PCSK9 secretion. PCSK9 expression is increased in the liver but secretion was reduced resulting in low plasma levels. The inventors have now identified PCSK9 deacetylation by SIRT1 as the mechanism that prevents its secretion and results in the accumulation of PCSK9 in the liver.

The data provided herein demonstrate that PCSK9 is acetylated by pCAF, thereby enhancing its secretion, while deacetylation of PCSK9 by SIRT1 reduces its hepatic secretion.

A limitation of the most widely used treatment for hypercholesterimia —the statins— is that they increase LDLR expression as well as PCSK9 secretion, thus reducing the efficiency of statins in reducing plasma cholesterol levels. Inhibition of PCSK9 by monoclonal antibodies is currently the only strategy known in the art to reduce plasma PCSK9 levels.

Modulation of PCSK9 acetylation status can therefore be used to alter PCSK9 plasma levels. Furthermore PCSK9 acetylation status can be used as a biomarker for the diagnosis and treatment of diseases involving lipid dysfunction like cardiovascular disease.

Materials and Methods

Cell Culture

Human HepG2 hepatocytes (American Type Culture Collection, Manassas, Va.) and murine RAW 264.7 cells (Mouse leukaemic monocyte macrophage cell line) were cultured in DMEM containing 10% fetal bovine serum (v/v). AML12 mouse hepatoma cells were cultured in a 1:1 (v/v) mixture of DMEM and Ham's F12 medium supplemented with insulin (5 μg ml-1), transferrin (5 μg ml-1), selenium (5 ng ml-1), dexamethasone (40 ng ml-1), and 10% fetal bovine serum (v/v). Where indicated, AML12 cells were exposed to 10 μM SRT3025 in 1% DMSO (v/v). The cells were maintained in a humidified atmosphere with 5% CO2 at 37° C.

Expression Vectors

Point mutations of lysine at position 421 of wild-type PCSK9 vector (Origene) was generated using Q5 Site-directed mutagenesis kit (New England Biolabs) by replacing lysine to arginine to mimic deacetylation. 2 μg of empty vector, wild-type PCSK9 and K421R vectors were transfected in HepG2 cells using Lipofectamine 2000.

Formaldehyde Cross-Linking

HepG2 cells were trypsinized and pelleted in a 50 ml reaction tube, re-suspended in PBS and counted. Cells were centrifuged again and re-suspended to 1×10⁷ cells/ml in formaldehyde solution. Cell pellets were incubated with formaldehyde for 3 minutes. The supernatant was removed and the reaction was quenched with 0.5 ml ice-cold 1.25 M glycine/PBS. Cells were washed once in 1.25 M glycine/PBS and lysed in 1 ml RIPA buffer (50 mM Tris HCl, pH 8.0, 150 mM sodium chloride, 1% NP40, 0.5% sodium deoxycholate, 0.1% SDS, 1 mM EDTA, protease inhibitors (Complete mini, EDTA-free, Roche Diagnostics) per cells for 5 minute on ice. After 30 minutes, cell lysates were treated with 50 strokes using a Dounce homogenizer. Lysates were spun for 30 minutes at 13300rpm to remove cell debris.

Immunoprecipitation and Western Blotting

PCSK9 was immunoprecipitated from human plasma (50 μL) using a sheep anti-human PCSK9 antibody (1:200) (RnD systems) and activated agarose beads coupled with goat anti-rabbit IgGs. Immunoprecipitated proteins were then eluted with 1.25M glycine and incubated with Laemmeli buffer at 95 degrees celcius for 5 mins and separated on 10% SDS-PAGE and transferred to a nitrocellulose membrane. Immunoblotting was carried out using the same anti-rabbit PCSK9 antibody (1:3000) (Abcam 125251) and the HRP-conjugated secondary sheep antibody. The blots were revealed by chemiluminescence.

Fluorogenic Peptide Deacetylation Assay

To analyse the deacetylation activity of SIRT1 on PCSK9, an acetylated PCSK9 peptide was designed with acetylated lysine 421 (Lys421)-tagged to an AMC fluorophore. The acetyl-PCSK9-AMC peptide was dissolved in DMSO and incubated with 1 μg SIRT1 with 1 μM, 10 μM, 100 μM and 1 mM SIRT1 activator SRT1720 in the deacetylation assay buffer containing 50 mM Tris-HCl [pH8.0], 1 mM MgCl2, 125 mM NaCl, 2.5 mM potassium chloride and 500 μM NAD+. Reactions were allowed to proceed for an incubation period of 1 hr at 37° C. Prior to quenching the reaction, 2 mM nicotinamide was added to 1× developing buffer (BML-KI176-1250, Enzo Lifesciences) in the deacetylation assay buffer (50 mM Tris-HCl [pH8.0], 1 mM MgCl2, 125 mM NaCl, 2.5 mM potassium chloride). At each time point, 50 μL of the reaction was removed and mixed with 50 μL of the developer solution. The quenched samples were kept at 37° C. for 45 min prior to fluorescence reading. The release of the fluorescent AMC was measured using a Tecan M200 PRO fluorometric plate reader with an excitation 360 nM and detection of emitted light in the range of 450-480 nM. All reactions were normalized to control reactions in the absence of β-NAD (Ffinal=F+NAD−F-NAD).

Alternatively, the substrate peptide may be labeled with a fluorescent substance and a quenching substance. Then, the peptide is cleaved to allow the generation of fluorescence.

Acetylation and Deacetylation Assay

Substrate protein, PCSK9 was incubated with 2 μg of recombinant pCAF and 50 μM acetyl-CoA in 20 μL of acetylation buffer (250 mM Tris-HCl [pH9.0], 20 mM MgCl2, 250 mM NaCl, 2.5 mM DTT). Reaction mixtures were incubated at 37° C. for 1 hour. Reaction was stopped by freezing. 20 μL of thawed acetyl-PCSK9 mixture was incubated with 2 μg of SIRT1 in deacetylation buffer (250 mM Tris-HCl [pH9.0], 20 mM MgCl₂, 250 mM NaCl, 2.5 mM DTT, 5 mM NAD⁺). Reaction mixtures were incubated at 37° C. for 1 hour. Reaction was stopped by addition of Laemmli buffer and heated for 5 mins at 95° C. Samples were resolved by sodium dodecyl sulfate-polyacrylamide gel electrophoresis (SDS-PAGE). The reactions were analyzed by western blotting using anti-PCSK9 antibody (Abcam) to verify loading and anti-acetyl lysine (Cell signalling) to verify acetylation.

Generation of Acetylation-Specific PCSK9 Antibodies.

The PCSK9 peptide for acetyl-K421 was IHFSA[K(Ac)] (SEQ ID NO 001) where the bracketted lysine residues was acetylated. The peptides were injected into two rabbits each at the Laboratory animal services center at University of Zurich and were boosted three times. We obtained high-titer and high-specificity antisera to detect PCSK9 acetylated at the appropriate lysine.

Similarly, polyclonal antibodies were raised against the remaining detected acetylation sites Lys273, Lys69, Lys83, Lys243, Lys258, Lys494, Lys506 using the following peptides:

All sequence positions are given with reference to the human PCSK9 sequence (SEQ ID NO 010).

| Name | Peptide design | Lysine number | SEQ ID NO |
|---|---|---|---|
| PCS1 | FSA[K(Ac)] | Lys421 | 002 |
| PCS2 | LEFIR[K(Ac)] | Lys273 | 003 |
| PCS4 | FHRCA[K(Ac)] | Lys69 | 004 |
| PCS5 | YVVVL[K(Ac)] | Lys83 | 005 |
| PCS6 | DAGVA[K(Ac)] | Lys243 | 006 |
| PCS7 | LNCQG[K(Ac)] | Lys258 | 007 |
| PCS8 | FSRSG[K(Ac)] | Lys494 | 008 |
| PCS9 | EAQGG[K(Ac)] | Lys506 | 009 |

Sequence positions are given with reference to the human PCSK9 sequence (SEQ ID NO 010).

Mass Spectrometry

Excised gel bands were cut into approximately 1 mm³ pieces. Gel pieces were washed twice with 100 mM ammonium bicarbonate/50% acetonitrile for 15 min at 50° C. and dehydrated with acetonitrile for 10 min. All supernatants were discarded. Rehydration of the gel pieces was with 10 mM Tris-HCl, 2 mM CaCl₂, pH 8.2 containing 5 ng/μl proteomics-grade recombinant trypsin (Roche Diagnostics, Mannheim, Germany) at 4° C. Microwave assisted digestion (Model Discover, CEM, Matthews, N.C.) was performed for 30 min at 5 W and 60° C. The supernatant was extracted and gel pieces were washed with 150 μl 0.1% trifluoroacetic acid/50% acetonitrile. All supernatants were combined and evaporated to dryness in with a SpeedVac concentrator. Digested samples were re-solubilized in 20 μL of 0.1% formic acid and were analyzed by either MALDI time-of-flight tandem mass spectrometry MALDI-TOF-TOF) or liquid chromatography electrospray tandem mass spectrometry (LC/MS/MS For Maldi-TOF-TOF 1 μl of the sample was mixed with 1 μl of matrix solution (0.7 mg/ml α-cyano-4-hydroxycinnamic acid in 0.1% trifluoroacetic acid/85% acetonitrile, 1 mM NH₄H₂PO₄) and spotted on the target, desalted and concentrated by washing the spot with 0.1% trifluoroacetic acid. Maldi spectra were acquired on an UltrafleXtreme (Bruker, Bremen, Germany). LC/MS/MS analyses were run on a nanoAcquity UPLC (Waters Inc.) connected to a Q Exactive mass spectrometer (Thermo Scientific) equipped with a Digital PicoView source (New Objective). An aliquot of 2 μL was injected. Peptides were trapped on a Symmetry C18 trap column (5 μm, 180 μm×20 mm, Waters Inc.) and separated on a BEH300 C18 column (1.7 μm, 75 μm×150 m, Waters Inc.) at a flow rate of 250 nl/min using a gradient from 1% solvent B (0.1% formic acid in acetonitrile, Romil)/99% solvent A (0.1% formic acid in water, Romil) to 40% solvent B/60% solvent A within 90 min. Full scan MS spectra were acquired in positive profile mode from 350-1500 m/z with an automatic gain control target of 3e6, an Orbitrap resolution of 70′000 (at 200 m/z), and a maximum injection time of 100 ms. The 12 most intense multiply charged (z≧+2) precursor ions from each full scan were selected for higher-energy collisional dissociation fragmentation with a normalized collision energy of 25 (arbitrary unit). Generated fragment ions were scanned with an Orbitrap resolution of 35′000 (at 200 m/z) an automatic gain control value of 1e5 and a maximum injection time of 120 ms. The isolation window for precursor ions was set to 2.0 m/z and the underfill ratio was at 3.5% (refereeing to an intensity threshold of 2.9e4). Each fragmented precursor ion was set onto the dynamic exclusion list for 40 s.

Peptides were identified by aligning the Maldi-TOF-TOF data with the known sequence of PCSK9 using the BioTools program (Bruker, Bremen, Germany) or by searching the SwissProt database (version 2015_11, 549832 entries) using the Mascot search engine (Matrix Science, version 2.4.1). Mascot was set up to search the SwissProt database assuming the digestion enzyme trypsin. Mascot was searched with a fragment ion mass tolerance of 0.030 Da and a parent ion tolerance of 10.0 PPM. Oxidation of methionine and acetylation of lysine was specified in Mascot as a variable modification. Scaffold (version Scaffold_4.4.8, Proteome Software Inc.) was used to validate MS/MS based peptide and protein identifications. Peptide identifications were accepted if they achieved an FDR less than 0.1% by the Scaffold Local FDR algorithm. Protein identifications were accepted if they achieved an FDR less than 1.0% and contained at least 2 identified peptides.

### Example 1

### In Vitro Acetylation and Deacetylation of PCSK9

Reagents for Acetylation Assay
Human PCSK9 full length protein (ab155727)
Active human PCSK9 full length protein (ab182697)
Acetyl coenzyme A sodium SigmaAldrich (A2056)
p300 SigmaAldrich (SRP8022)
pCAF Histone Acetyltransferase Cayman Chemical (10009115)

Buffers for Acetylation:

HAT Buffer:

250 mM Tris-HCl [pH9.0], 20 mM MgCl₂, 250 mM NaCl, 2.5 mM DTT 5×SDAC Buffer

250 mM Tris-HCl [pH9.0], 20 mM MgCl₂, 250 mM NaCl, 2.5 mM DTT, 5 mM NAD⁺,

Add 1 mM Sodium Butyrate (only for positive control)

Protocol for In Vitro pCAF Acetylation Assay
1. Prepare 1×HAT Buffer (250 mM Tris-HCl [pH9.0], 20 mM MgCl₂, 250 mM NaCl, 2.5 mM DTT)
2. Add the following components in 1.5 ml tube:
   a) Substrate 20 μg
   b) Recombinant pCAF protein 2 μg
   c) Acetyl-CoA 100 μM
   d) 1× SDAC buffer
   e) Incubate 37° C., 1 hour
   f) Stop the reaction by adding 5×SDS sample buffer

Protocol for In Vitro SIRT1 Deacetylation Assay
1. Prepare 1×SDAC Buffer (250 mM Tris-HCl [pH9.0], 20 mM MgCl₂, 250 mM NaCl, 2.5 mM DTT, 5 mM NAD⁺)
2. Add the following components in 1.5 ml tube:
   g) Acetylated substrate 10 μg
   h) SIRT1 2 μg
   i) 1× SDAC buffer
   j) Incubate 37° C., 1 hour
   k) Stop the reaction by adding 5×SDS sample buffer.

### Example 2

### Detection of PCSK9 Acetylation

Procedure:

Samples dissolved in electrophoresis sample buffer were loaded on a gel.

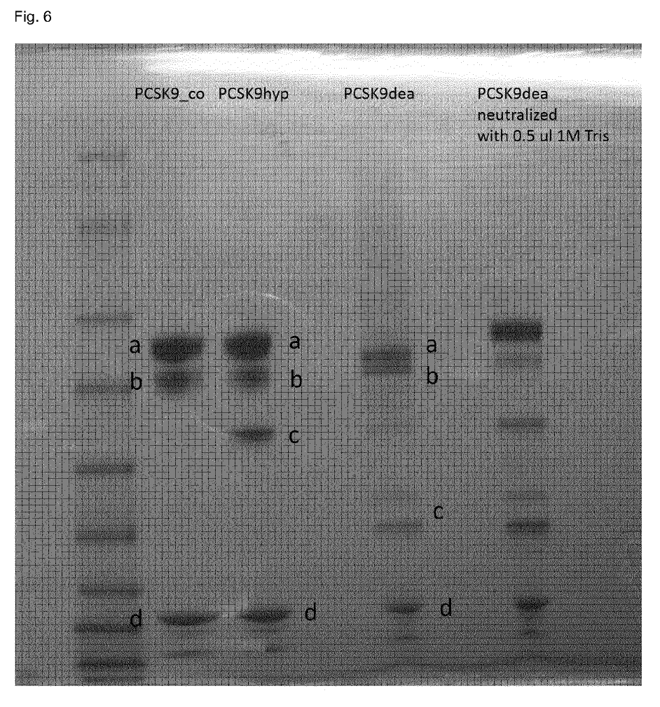

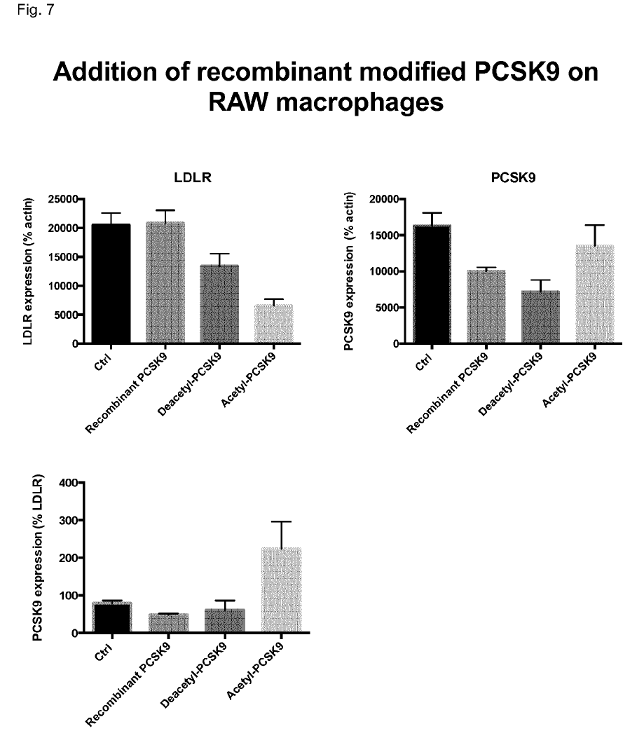

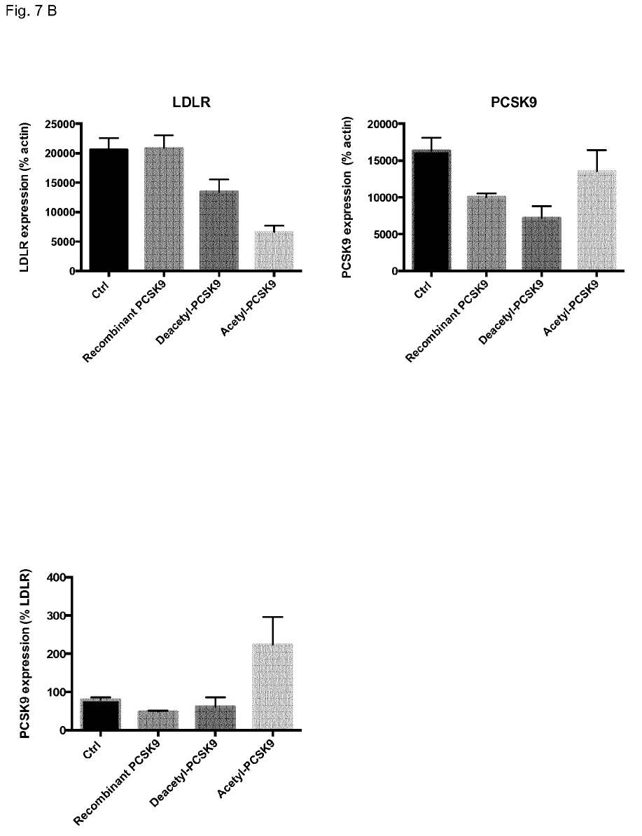

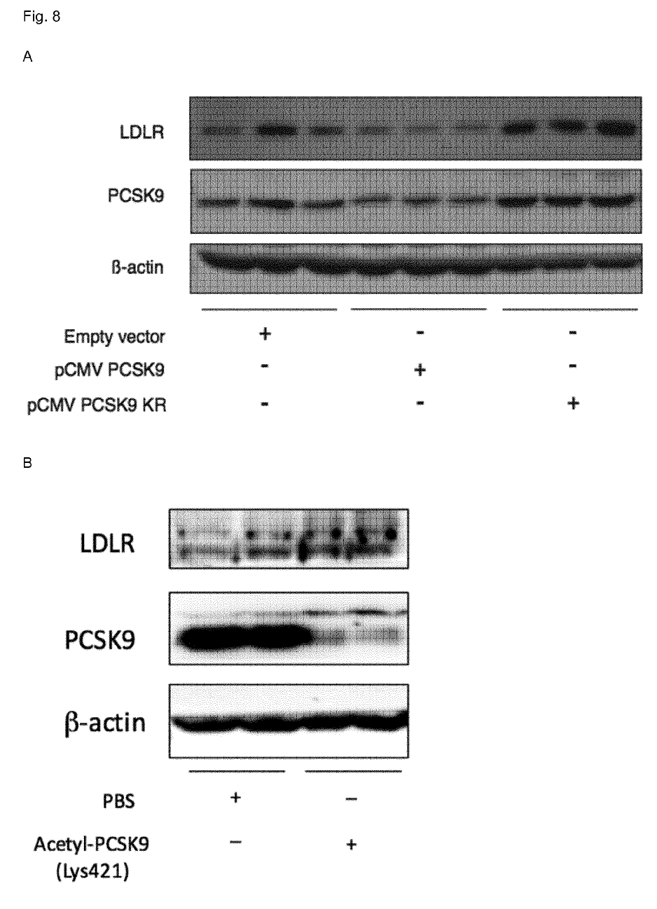

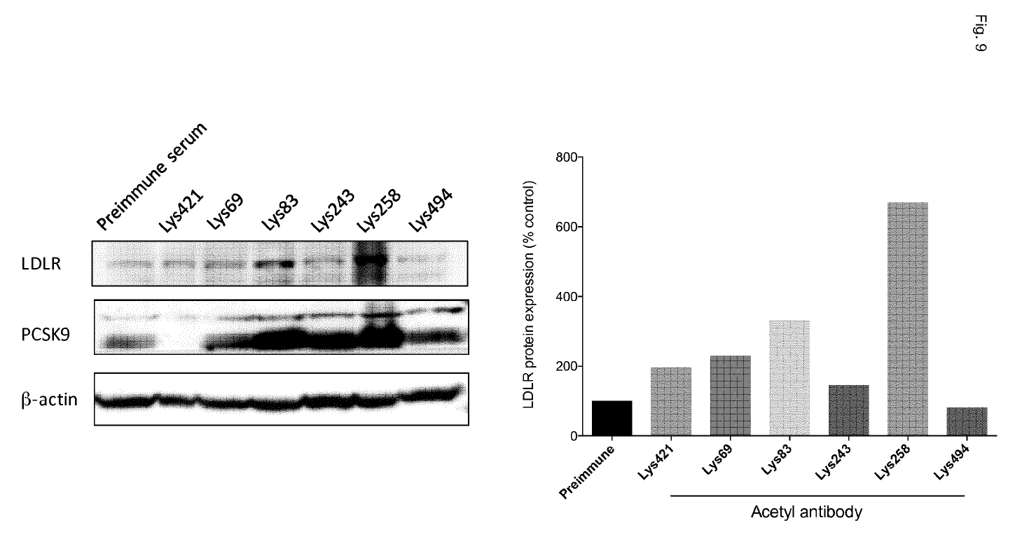

Test gel (Bis-Tris gradient gel 4-12%, MOPS buffer): 10 μl of each sample loaded. The sample PCSK9dea was loaded after neutralization with 0.5 μl 1 M Tris pH 8.2. (FIG. 6)

Preparative gel (Bis-Tris gradient gel 4-12%, MOPS buffer): same as for the test gel, but 30 μl were loaded.
Bands a and b of each lane and band c of PCSK9hyp (FIG. 6) were cut in small pieces.
Gel pieces were washed twice with 100 μl 100 mM NH₄HCO₃/50% acetonitrile and washed once with 50 μl acetonitrile.
All three supernatants were discarded.
To the samples were added:
   +10 μl trypsin (5 ng/μl in 10 mM Tris, 2 mM CaCl₂, pH 8.2)
   +30 μl buffer (10 mM Tris, 2 mM CaCl₂, pH 8.2)
Microwave treatment for 30 min at 60° C.
Supernatant was removed and gel pieces extracted with 150 μl 0.1% TFA/50% acetonitrile.
All supernatants were combined and dried.
Samples (except PCSK9hyp_c) were dissolved in 15 μl 0.1% TFA. 1 μl was mixed with matrix and the samples were measured by MALDI.
Data were aligned with the sequence of hPCSK9.

Remaining sample were dried, dissolved in 20 μl 0.1% formic acid and transferred to autosampler vials for LC/MS/MS.

1 μl (bands a and b of PCSK9_co and hyp, 2 μl (band PCSK9hyp_c), and 3 μl (bands PCSK9dea_a and _b) were injected.

Database searches were performed by using the Mascot (SwissProt, all species) and Peaks (search against a database containing only the sequence of PCSK9) search programs. Mascot search results are summarized in Scaffold (if present, trypsin, keratin, other common contaminants, and decoy hits are hidden).

### Example 3

### Mapping of PCSK9 Lysine Acetylation by Tandem Mass Spectrometry

PCSK9 was immunoprecipitated from HepG2 cells were incubated with recombinant SIRT1 with and without NAD⁺. The protein from the Colloidal Coomassie stained band was digested with trypsin and the resulting peptides were analyzed by LC-MS/MS. MS/MS data were analyzed using the sequences of PCSK9 with the SEQUEST algorithm, allowing the detection of acetylated lysine residues (see FIG. 5 and sequences below). FIG. 5A shows all peptides recovered in the LC-MS/MS experiment for PCSK9. FIGS. 5B-D show the identification of acetylation at K421, K273 & K506 in PCSK9 by MALDI.

The following sequences were determined (underlined K refer to acetylated lysine residues, numbers to positions in the human PCSK9 sequence (SEQ ID NO 10)):

| | |
|---|---|
| SEQ ID NO 011: | 492-510 SGKRRGERMEAQGGKLVCR |
| SEQ ID NO 012: | 238-272 DAGVAKGASMRSLRVLNCQGKGTVSGTLIGLEFIR |
| SEQ ID NO 013 | 223-258 CDSHGTHLAGVVSGRDAGVAKGASMRSLRVLNCQGK |
| SEQ ID NO 014 | 67-97 CAKDPWRLPGTYVVVLKEETHLSQSERTARR |
| SEQ ID NO 015 | 411-425 LRQRLIHFSAKDVINE |
| SEQ ID NO 016 | 271-300 IRKSQLVQPVGPLVVLLPLAGGYSRVLNAA |

1. A ligand, particularly an isolated antibody, that binds to acetylated PCSK9.

2. The ligand of claim 1, wherein binding of said antibody to acetylated PCSK9 is characterized by a first dissociation constant KD1, and binding of said antibody to non-acetylated PCSK9 is characterized by a second dissociation constant KD2, and the relation of KD1 and KD2 is KD1*100<KD2.

3. The ligand of claim 2, wherein KD1<10 nmol/l, KD1<1 nmol/l, or KD1<0.1 nmol/l.

4. The ligand of claim 1, wherein the ligand binds to an epitope on acetylated PCSK9, said epitope comprising:
a. an acetylated lysine residue Lys421;
b. an acetylated lysine residue Lys273;
c. an acetylated lysine residue Lys69;
d. an acetylated lysine residue Lys83;
e. an acetylated lysine residue Lys243;
f. an acetylated lysine residue Lys258;
g. an lysine residue Lys494; and/or
h. an acetylated lysine residue Lys506.

5. The ligand of claim 1, wherein the ligand is a human antibody, a humanized antibody or a chimeric antibody.

6. A pharmaceutical composition comprising the ligand binding to acetylated PCSK9 of claim 1 and a pharmaceutically acceptable carrier.

7. (canceled)

8. A method of reducing LDL-cholesterol level in a patient in need thereof, said method comprising administering to the subject an effective amount of the ligand binding to acetylated PCSK9 of claim 1.

9. A method of treating a cholesterol related disorder in a patient in need thereof, said method comprising administering to the subject an effective amount of the ligand binding to acetylated PCSK9 of claim 1.

10. A method of treating hypercholesterolemia in a patient in need thereof, said method comprising administering to the subject an effective amount of the ligand binding to acetylated PCSK9 of claim 1.

11. A nucleic acid molecule encoding the ligand according to claim 1.

12. An expression cassette comprising the nucleic acid molecule of claim 11 under control of a promoter sequence operable in a mammalian cell.

13. A transgenic cell comprising an expression cassette according to claim 12.

14. A cell able to produce a ligand, particularly an antibody according to claim 1.

15. An isolated acetylated PCSK9 molecule.

16. The acetylated PCSK9 molecule according to claim 15, wherein a position of acetylation is selected from Lys421, Lys273, Lys69, Lys83, Lys243, Lys258, Lys494, and Lys506.

17. An immunogen comprising an acetylated-PCSK9 peptide, particularly an acetylated-PCSK9 peptide acetylated in position Lys421, linked to an immunogenic carrier.

18. The immunogen according to claim 17, wherein the immunogenic carrier is selected from Diphtheria toxiod, CRM197 or a VLP selected from HBcAg, HBsAg, Qbeta, PP7, PPV or Norwalk Virus VLP.

19. The ligand of claim 1, wherein the ligand does not bind to non-acetylated PCSK9.

20. The method of claim 8, wherein reducing LDL-cholesterol level in the patient treats atherosclerosis.


SEQUENCE LISTING (16 sequences)

SEQ ID NO: 1 - protein, 6 aa, Homo sapiens
  MOD_RES (6)..(6): ACETYLATION, acetylated lysine residue corresponds to Lys421 of human PCSK9
IHFSAK

SEQ ID NO: 2 - protein, 4 aa, Homo sapiens
  MOD_RES (4)..(4): ACETYLATION, acetylated lysine residue corresponds to Lys421 of human PCSK9
FSAK

SEQ ID NO: 3 - protein, 6 aa, Homo sapiens
  MOD_RES (6)..(6): ACETYLATION, acetylated lysine residue corresponds to Lys273 of human PCSK9
LEFIRK

SEQ ID NO: 4 - protein, 6 aa, Homo sapiens
  MOD_RES (6)..(6): ACETYLATION, acetylated lysine residue corresponds to Lys69 of human PCSK9
FHRCAK

SEQ ID NO: 5 - protein, 6 aa, Homo sapiens
  MOD_RES (6)..(6): ACETYLATION, acetylated lysine residue corresponds to Lys83 of human PCSK9
YVVVLK

SEQ ID NO: 6 - protein, 6 aa, Homo sapiens
  MOD_RES (6)..(6): ACETYLATION, acetylated lysine residue corresponds to Lys243 of human PCSK9
DAGVAK

SEQ ID NO: 7 - protein, 6 aa, Homo sapiens
  MOD_RES (6)..(6): ACETYLATION, acetylated lysine residue corresponds to Lys258 of human PCSK9
LNCQGK

SEQ ID NO: 8 - protein, 6 aa, Homo sapiens
  MOD_RES (6)..(6): ACETYLATION, acetylated lysine residue corresponds to Lys494
FSRSGK

SEQ ID NO: 9 - protein, 6 aa, Homo sapiens
  MOD_RES (6)..(6): ACETYLATION, acetylated lysine residue corresponds to Lys506 of human PCSK9
EAQGGK

SEQ ID NO: 10 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 11 - protein, 19 aa, Homo sapiens
  MOD_RES (3)..(3): ACETYLATION, acetylated lysine residue corresponds to Lys494 of human PCSK9
  MOD_RES (15)..(15): ACETYLATION, acetylated lysine residue corresponds to Lys506 of human PCSK9
SGKRRGERMEAQGGKLVCR

SEQ ID NO: 12 - protein, 35 aa, Homo sapiens
  MOD_RES (6)..(6): ACETYLATION, acetylated lysine residue corresponds to Lys243 of human PCSK9
  MOD_RES (21)..(21): ACETYLATION, acetylated lysine residue corresponds to Lys258 of human PCSK9
DAGVAKGASMRSLRVLNCQGKGTVSGTLIGLEFIR

SEQ ID NO: 13 - protein, 36 aa, Homo sapiens
  MOD_RES (21)..(21): ACETYLATION, acetylated lysine residue corresponds to Lys243 of human PCSK9
  MOD_RES (36)..(36): ACETYLATION, acetylated lysine residue corresponds to Lys258 of human PCSK9
CDSHGTHLAGVVSGRDAGVAKGASMRSLRVLNCQGK

SEQ ID NO: 14 - protein, 31 aa, Homo sapiens
  MOD_RES (3)..(3): ACETYLATION, acetylated lysine residue corresponds to Lys69 of human PCSK9
  MOD_RES (17)..(17): ACETYLATION, acetylated lysine residue corresponds to Lys83 of human PCSK9
CAKDPWRLPGTYVVVLKEETHLSQSERTARR

SEQ ID NO: 15 - protein, 16 aa, Homo sapiens
  MOD_RES (11)..(11): ACETYLATION, acetylated lysine residue corresponds to Lys421 of human PCSK9
LRQRLIHFSAKDVINE

SEQ ID NO: 16 - protein, 30 aa, Homo sapiens
  MOD_RES (3)..(3): ACETYLATION, acetylated lysine residue corresponds to Lys273 of human PCSK9
IRKSQLVQPVGPLVVLLPLAGGYSRVLNAA